(19) United States
(12) Patent Application Publication
(10) Pub. No.: US 2014/0371608 A1
(43) Pub. Date: Dec. 18, 2014

(54) STIMULATIVE ELECTROTHERAPY USING AUTONOMIC NERVOUS SYSTEM CONTROL

(71) Applicant: Dyansys, Inc., San Mateo, CA (US)

(72) Inventor: Srini NAGESHWAR, Los Gatos, CA (US)

(73) Assignee: Dyansys, Inc., San Mateo, CA (US)

(21) Appl. No.: 13/917,471

(22) Filed: Jun. 13, 2013

(51) Int. Cl. A61B 5/00; A61N 1/36; A61B 5/024

(52) U.S. Cl. CPC A61B 5/4035; A61B 5/024; A61B 5/7282; A61B 5/7278; A61B 5/4836; A61N 1/36139
USPC 600/508; 600/300; 607/62

(57) ABSTRACT

Methods for caring for a patient are disclosed. In some embodiments, the methods include measuring a first autonomic nervous system condition of the patient, calculating a first autonomic dysfunction based on the measured first autonomic nervous system condition, and calculating a first sympathovagal balance based on the first measured autonomic nervous system condition. The method also includes treating the patient, measuring a second autonomic nervous system condition of the patient, calculating a second sympathovagal balance based on the second measured autonomic nervous system condition, and comparing the second sympathovagal balance with the first sympathovagal balance. The method also includes calculating a second autonomic dysfunction based on the measured second autonomic nervous system condition, and comparing the second autonomic dysfunction with the first autonomic dysfunction.

### CROSS-REFERENCES TO RELATED APPLICATIONS

This application is related to U.S. Pat. No. 7,092,849, titled “EXTRACTING CAUSAL INFORMATION FROM A CHAOTIC TIME SERIES,” granted Aug. 15, 2006, the content of which is incorporated herein by reference in its entirety. This application is also related to the following applications filed herewith: U.S. Patent Application Attorney Docket No. 89562-000400US-874044, titled “METHOD AND APPARATUS FOR AUTONOMIC NERVOUS SYSTEM SENSITIVITY-POINT TESTING”, U.S. Patent Application Attorney Docket No. 89562-000500US-874022, titled “COMPUTER IMPLEMENTED TRAINING OF A PROCEDURE,” and Attorney Docket No. 89562-001000US-876815, titled “METHOD AND APPARATUS FOR STIMULATIVE ELECTROTHERAPY,” the contents of all of which are incorporated herein by reference in their entirety.

### FIELD OF THE INVENTION

The present invention generally pertains to a method and apparatus for extracting information from a chaotic time series of data generated based on the autonomic nervous system of a patient, and using the information to enhance therapy administered to the patient. More precisely, the present invention pertains to a method and apparatus for analyzing the state of a patient before and after treatment.

### BACKGROUND OF THE INVENTION

The autonomic nervous system (ANS), with its sympathetic and parasympathetic subsystems, governs involuntary actions of the cardiac muscle and every visceral organ in the body. The ANS is not directly accessible to voluntary control. Instead, it operates in an autonomic fashion on the basis of autonomic reflexes and central control. One of its major functions is the maintenance of homeostasis within the body. The ANS further plays an adaptive role in the interaction of the organism with its surroundings.

Heart rate variability has been shown to be a powerful means of assessing the influence of the ANS on the cardiac system. Heart rate variability is therefore a powerful indicator of the state of the ANS, and can be used as an effective means of assessing the state of physiological conditions related to the ANS, such as chronic pain.

In many diseases, the sympathetic and/or parasympathetic subsystems of the ANS are affected, leading to autonomic dysfunction. It is then important to have reliable and representative measures of the activity and the state of the ANS.

Three main classes of methods are used to recover information about the ANS from the heart rate variability: spectral analysis (also called time domain analysis), statistics and calculation of a correlation dimension (or any related dimension). These methods do not give easy interpretable outcomes. Moreover, they lack reliability and are often not mathematically appropriate in their considered application.

Without reliable and representative measures of the ANS, effects of treatment for certain conditions can be measured only subjectively. For example, to measure pain, a patient may be asked to rate their pain level on a scale of 1-10.

### BRIEF SUMMARY OF THE INVENTION

One inventive aspect is a method of caring for a patient. The method includes measuring a first autonomic nervous system condition of the patient, calculating a first autonomic dysfunction based on the measured first autonomic nervous system condition, and calculating a first sympathovagal balance based on the first measured autonomic nervous system condition. The method also includes treating the patient, measuring a second autonomic nervous system condition of the patient, calculating a second sympathovagal balance based on the second measured autonomic nervous system condition, and comparing the second sympathovagal balance with the first sympathovagal balance. The method also includes calculating a second autonomic dysfunction based on the measured second autonomic nervous system condition, and comparing the second autonomic dysfunction with the first autonomic dysfunction.

Another inventive aspect is a method of caring for a patient. The method includes measuring a first autonomic nervous system condition of the patient, calculating a first sympathovagal balance based on the first measured autonomic nervous system condition, and treating the patient. The method also includes measuring a second autonomic nervous system condition of the patient, calculating a second sympathovagal balance based on the second measured autonomic nervous system condition, and comparing the second sympathovagal balance with the first sympathovagal balance.

Another inventive aspect is a method of caring for a patient. The method includes measuring a first autonomic nervous system condition of the patient, calculating a first autonomic dysfunction based on the measured first autonomic nervous system condition, and treating the patient. The method also includes measuring a second autonomic nervous system condition of the patient, calculating a second autonomic dysfunction based on the measured second autonomic nervous system condition, and comparing the second autonomic dysfunction with the first autonomic dysfunction.

### BRIEF DESCRIPTION OF THE DRAWINGS

FIG. 1 is a flowchart illustrating a method of caring for a patient.

FIG. 2A is a flowchart illustrating a method of calculating autonomic dysfunction, which can be used in the method of FIG. 1. FIG. 2B illustrates Plot 1, which is an example of a set of sorted difference values.

FIG. 3 is a flowchart illustrating a method of treating a patient, which can be used in the method of FIG. 1.

FIG. 4 is a chart which can be used to determine a parameter value for use in the method of FIG. 3 based on a measured characteristic of the ANS of the patient.

### DETAILED DESCRIPTION OF THE INVENTION

Particular embodiments of the invention are illustrated herein in conjunction with the drawings.

Various details are set forth herein as they relate to certain embodiments. However, the invention can also be implemented in ways which are different from those described herein. Modifications can be made to the discussed embodiments by those skilled in the art without departing from the invention. Therefore, the invention is not limited to particular embodiments disclosed herein.

Particular biological events produced by a patient are governed by the ANS of the patient. Thus, a condition of the ANS of the patient may be determined through appropriate analysis of data representing the particular events. Furthermore, because the condition of the ANS of the patient may be related to one or more conditions for which the patient may seek treatment, the analysis of the data representing the biological events may be used as a quantitative measurement of the one or more conditions.

For example, the biological events may be related to the cardiac system of the patient. Thus, data representing heart rate or heart rate variability of the patient may be used to determine a measurement of pain experienced by the patient. Additionally or alternatively the biological events may be related to the respiratory system or to brain activity of the patient.

In some embodiments, conditions correlated with the biological events include one or more of chronic pain, anxiety, depression, and sleep problems.

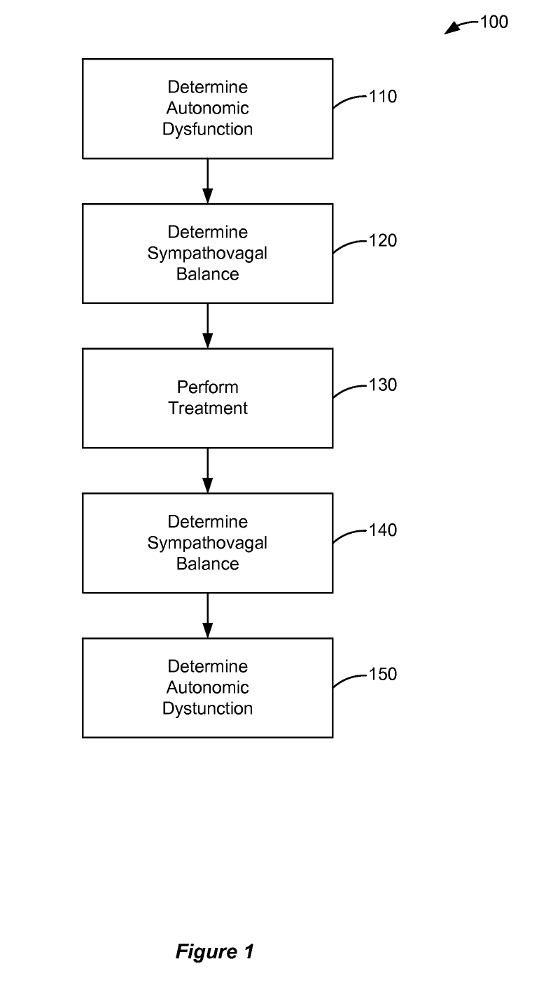

FIG. 1 is a flowchart illustrating a method 100 of caring for a patient. The patient may be seeking treatment for one or more conditions which may be measured through analysis of data related to biological events governed by the ANS of the patient. For example, the patient may be experiencing chronic pain.

According to the method 100, before treatment, autonomic dysfunction and Sympathovagal balance are determined. In addition, following treatment, autonomic dysfunction and Sympathovagal balance are again determined. A difference between before and after values of the autonomic dysfunction and Sympathovagal balance of the patient may be used as an indication of the efficacy of the treatment.

In step 110, autonomic dysfunction is determined.

In some embodiments, one or more methods and/or systems described in appendix 1 is used to determine autonomic dysfunction. For example, data representing biological events produced by the patient, which are governed by the ANS of the patient may be recorded using an apparatus described in appendix 1. In addition, one or more data analysis methods and systems described in appendix 1 may be used to calculate an autonomic dysfunction of the patient based on the recorded biological event data.

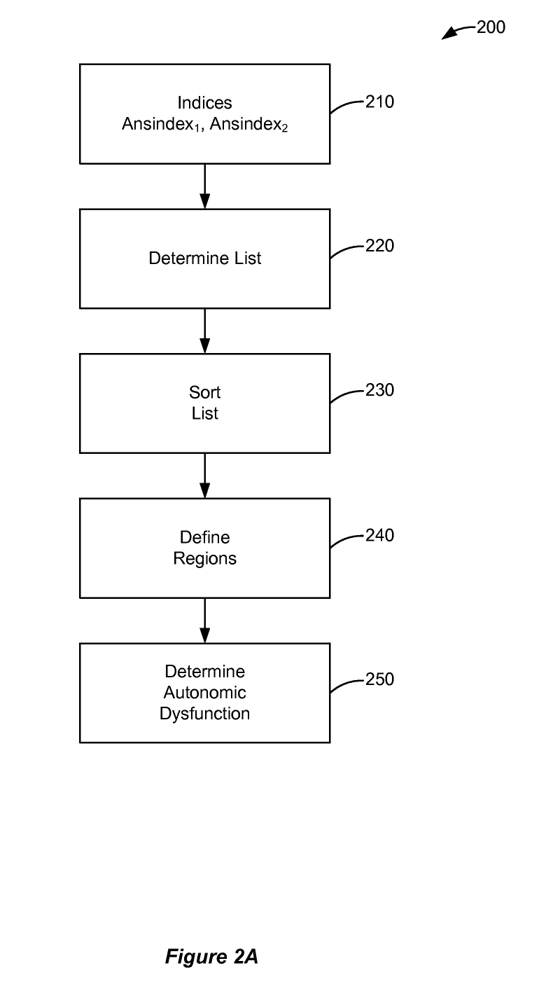

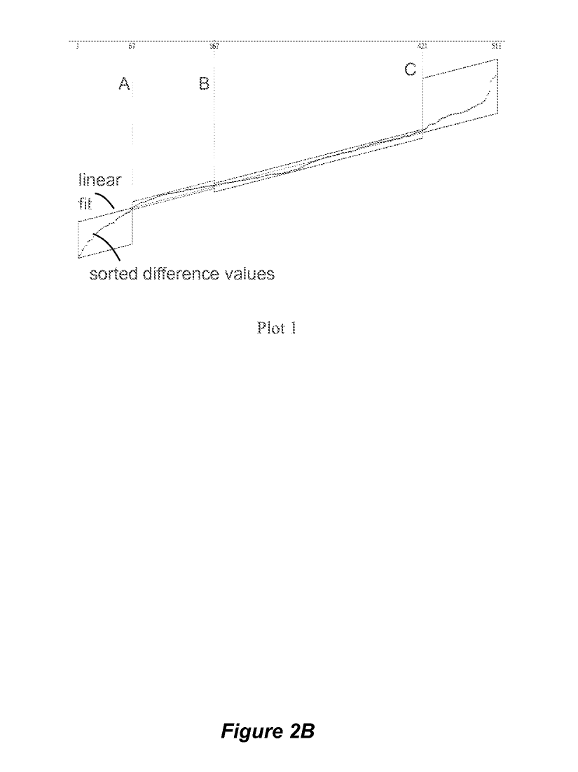

In some embodiments, methods and/or systems not described in the appendix 1 may be used to the autonomic dysfunction of the patient. For example, a method of determining an autonomic dysfunction of the patient described below with reference to FIG. 2A may be used.

In step 120, Sympathovagal balance is determined.

In some embodiments, one or more methods and/or systems described in appendix 1 is used to determine Sympathovagal balance. For example, data representing biological events produced by the patient, which are governed by the ANS of the patient may be recorded using an apparatus and/or method described in appendix 1. In addition, one or more data analysis methods and systems described in appendix 1 may be used to calculate a Sympathovagal balance of the patient based on the recorded biological event data. In some embodiments, the recorded biological event data used to calculate the autonomic dysfunction of the patient is also used to calculate the Sympathovagal balance of the patient.

In some embodiments, a balance curve is calculated using one or more methods and systems described in appendix 1, and Sympathovagal balance is determined based on one or more parameters extracted from balance curve. For example, one or more of the minimum, the maximum, the midpoint, the mean, and the median for either the horizontal or vertical axis values may be used as the Sympathovagal balance. Additionally or alternatively, the presence of loops or upholding of long flat transitions may be used as the Sympathovagal balance.

In some embodiments, methods and/or systems not described in the appendix 1 may be used to the Sympathovagal balance of the patient.

In step 130, a treatment is performed on the patient. In some embodiments, the treatment comprises providing electrical stimulus to selected sites on the body of the patient. Alternatively, one or more other treatments may be performed on the patient. For example, physical therapy, other forms of stimulation, manipulation, and pain medication, such as opioids.

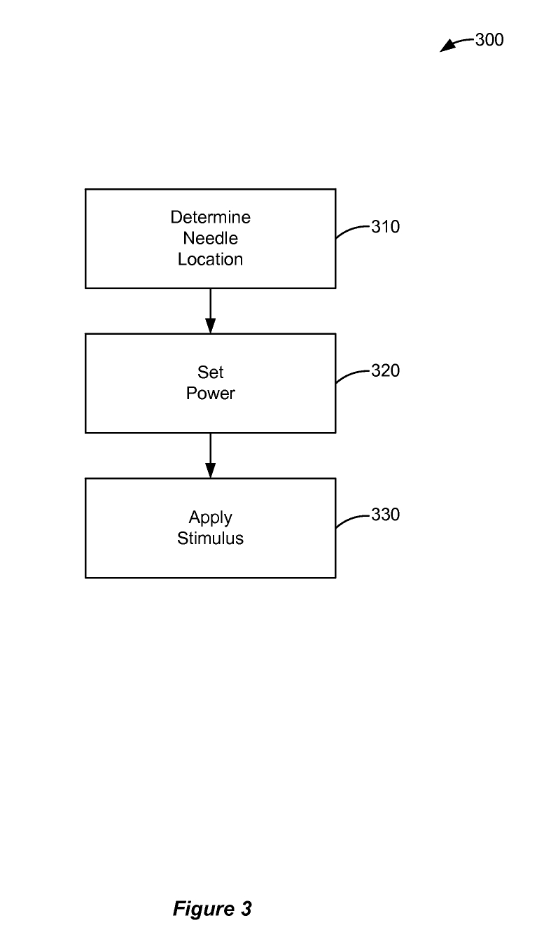

In some embodiments, a method of treating the patient described below with reference to FIG. 3 may be used.

In step 140, following the treatment, Sympathovagal balance of the patient is again determined. The Sympathovagal balance determined after the treatment may be compared with the Sympathovagal balance determined prior to the treatment. The comparison may be used to judge efficacy of the treatment.

In some embodiments, in step 140, the Sympathovagal balance of the patient is determined using systems and methods substantially identical to the systems and methods used in step 120 to determine the Sympathovagal balance of the patient prior to the treatment. In some embodiments, the methods and systems used in step 140 to determine the Sympathovagal balance of the patient after the treatment may be different from the methods and systems used in step 120 to determine the Sympathovagal balance of the patient prior to the treatment.

In step 150, following the treatment, an autonomic dysfunction of the patient is again determined. The autonomic dysfunction determined after the treatment may be compared with the autonomic dysfunction determined prior to the treatment. The comparison may be used to judge efficacy of the treatment.

In some embodiments, in step 150, the autonomic dysfunction of the patient is determined using systems and methods substantially identical to the systems and methods used in step 110 to determine the autonomic dysfunction of the patient prior to the treatment. In some embodiments, the methods and systems used in step 150 to determine the autonomic dysfunction of the patient after the treatment may be different from the methods and systems used in step 110 to determine the autonomic dysfunction of the patient prior to the treatment.

In some embodiments, the method of FIG. 1 is repeated. For example, the method of FIG. 1 may be used in a first treatment session. As part of the first treatment session, an efficacy of the first treatment may be judged based on the comparisons of the autonomic dysfunction and Sympathovagal balance values before and after the first treatment. Likewise, the method of FIG. 1 may be used in a second treatment session. Similar to the first treatment session, as part of the second treatment session, an efficacy of the second treatment may be judged based on comparisons of the autonomic dysfunction and Sympathovagal balance values before and after the second treatment. In some embodiments, the second treatment session includes about 1, 2, 3, 4, 5, 6, 7, 8, 9, 10, 11, or 12 minutes, hours, days, weeks, months, or years after the first treatment session.

In addition, autonomic dysfunction and Sympathovagal balance values determined as part of the second treatment session may be be compared with autonomic dysfunction and Sympathovagal balance values determined as part of the second treatment session. Such a comparison may indicate efficacy of the treatment over multiple treatment sessions.

FIG. 2A is a flowchart illustrating a method 200 of calculating an autonomic dysfunction of a patient. The method 200 can be used, for example, in the method 100 illustrated in FIG. 1. In some embodiments, the method 200 illustrated in FIG. 2A is performed separately and distinct from the method 100 illustrated in FIG. 1. In addition, the method 100 illustrated in FIG. 1 may use a method of calculating autonomic dysfunction which is different from the method 200 illustrated in FIG. 2A.

According to the method 200, an autonomic dysfunction is calculated based on recorded data representing biological events which are governed by the ANS of the patient.

In step 210, a first index ANSindex1 and a second index ANSindex2, are calculated according to methods and systems described in appendix 1. In alternative embodiments, ANSindex1 and ANSindex2 may be calculated using different methods and systems. In some embodiments, ANSindex1 and ANSindex2 may be calculated in response to each of a plurality of successive biological events. For example, in response to each of a number of heartbeats as measured, for example, with an EKG, ANSindex1 and ANSindex2 values may be calculated. In some embodiments, ANSindex1 and ANSindex2 values may be calculated in response to each of a series of 400 heartbeats. In some embodiments, ANSindex1 and ANSindex2 values may be calculated in response to each of a series of 512 heartbeats. In some embodiments, the data from a certain number of heartbeats, for example 60, are used for calibration, or other purposes. In some embodiments, the heartbeats are successive.

In step 220, a set of difference values (DV) is calculated. Each difference value of the set is calculated based on the ANSindex1 and ANSindex2 values calculated in response to one of the successive biological events, as described with reference to step 210. For example, in step 210, for each of the successive biological events, an ANSindex1 value and an ANSindex2 value are calculated, and in step 220 a difference value between the ANSindex1 value and the ANSindex2 value for each successive biological event is calculated. The difference values calculated for all of the biological events forms the set of difference values.

For example, in some embodiments,

DVᵢ=ANSindex2ᵢ−ANSindex1ᵢ,

where i is an index indicating data points.

In step 230, the set of difference values is sorted. For example, the set of difference values may be sorted from lowest difference value to highest difference value. In other embodiments the second difference values may be sorted from highest difference value to lowest difference value.

FIG. 2B illustrates Plot 1, which is an example of a set of sorted difference values. The difference values are plotted in the sorted order, with the lower difference values being plotted to the left of the higher difference values, and where the distance from the horizontal axis corresponds with the value of each of the sorted difference values. Plot 1 also shows a linear fit reference line.

In step 240, the sorted difference values are separated into different regions. For example, four regions may be defined. Indicators A, B, and C identify boundaries between adjacent regions of the example set of difference values shown in Plot 1. In this example, the indicators A, B, and C align with difference values 67, 167, and 421, respectively. In some embodiments, the regions are determined based on the linearity or second derivative of the sorted difference values. For example, each region may include the difference values which correspond to points where the second derivative differs by less than a threshold. In some embodiments, regions may be determined by alternate crossing of a middle portion linear or cubic fit, and/or a distance within various thresholds to a linear or cubic fit.

Each of the regions may correspond with a certain characteristic of the ANS of the patient. For example, the first and last, lower and upper regions may correspond respectively to a profound altered state and a superficial transient change of autonomic function whereas the quasi-linear middle regions may indicate a melded durable state of autonomic homeostasis.

In step 250, information represented in the set of sorted difference values is used to calculate an autonomic dysfunction of the patient. Various mathematical methods may be used.

For example, a value V, may be determined for each of the four regions. In some embodiments, the value for each region is determined by summing the difference values of the region. Alternatively, the value for each region may be determined by summing the difference values of the region raised to an exponent. For example, the exponent may be 2, 3, 4, 5, or another value. In some embodiments, the exponent may not be a whole number, may be irrational, and/or may be negative. As a nonlimiting example, the value for each of the regions may be determined by summing the difference values of the region raised to the fourth power.

For example, in some embodiments,

Vᵣ=Σ₁²(DVᵢ)⁴,

where i is a summing index indicating data points in the region, n is the number of points in the region, and r identifies the region.

In some embodiments, the values for the regions are each multiplied by a coefficient (c) specific to the region associated therewith. For example, the value associated with the first region may be multiplied by a coefficient equal to −8.2045, the value associated with the second region may be multiplied by a coefficient equal to 1.769, the value associated with the third region may be multiplied by a coefficient equal to 0.90025, and the value associated with the fourth region may be multiplied by a coefficient equal to 1.903. Alternatively, the coefficient for the first region may be equal to −9.215, the coefficient for the second region may be equal to −530, the coefficient for the third region may be equal to 0.7, and the coefficient for the fourth region may be equal to 1.23. Other coefficient values may be used.

In some embodiments, the values multiplied by their respective coefficients are summed. Further, a constant C may be added to the summed values multiplied by their respective coefficients. For example, −2600 may be added to the summed values multiplied by their respective coefficients. Alternatively, the constant C may be equal to −1650.

In some embodiments, the coefficient values {a->−8-2045, b->1.769, c->0.90025, d->1.903, offset->−2600} are used with a lower sampling rate for the input EKG signal (for example, 300 Hz), and the coefficient values {a->−9.215, b->−530, c->0.7, d->1.23, offset->−1650 } are used with a higher sampling rate for the input EKG signal (for example, 600 Hz or 1.2 kHz).

To calculate the autonomic dysfunction AD, the result of the summing may be raised to an exponent equal to the inverse of the exponent used for determining the values associated with each region.

For example, in some embodiments,)

AD=(C+Σ₁ⁿcᵢVᵢ)^{1/4},

where i is a summing index indicating regions, and n is the number of regions.

In some embodiments, a value representing the calculated autonomic dysfunction is graphically shown on a display associated with an apparatus used for calculating the autonomic dysfunction.

FIG. 3 is a flowchart illustrating a method 300 of treating a patient. The method 300 can be used in the method 100 illustrated in FIG. 1. In some embodiments, the method 300 illustrated in FIG. 3 may be performed separately and distinct from the method 100 illustrated in FIG. 1. In addition, the method 100 illustrated in FIG. 1 may use a method of treating a patient which is different from the method 300 illustrated in FIG. 3. For example, physical therapy, other forms of stimulation, manipulation, and pain medication, such as opioids.

In the method 300, the patient is treated by electrically stimulating points on the patient's skin to which the autonomic nervous system is sensitive.

In step 310, locations on the patient's skin having autonomic nervous system sensitivity are identified. For example, a graphical representation of a least a portion of the patient's body having sensitivity points identified may be referenced. In some embodiments, the locations correspond with locations identified as acupuncture points.

In step 320, an electrical stimulus source generator is adjusted so as to provide an appropriate stimulus signal. For example, one or more parameters, such as at least one of a frequency, an amplitude, a DC offset, a power, and a treatment duration may be programmed into the electrical stimulus source generator. In some embodiments, the electrical stimulus source generator is programmed with a value determined based on a value calculated based on biological event data. For example, one or more values associated with autonomic dysfunction or Sympathovagal balance may be used to determine one or more values for the one or more parameters to be program into the electrical stimulus source generator.

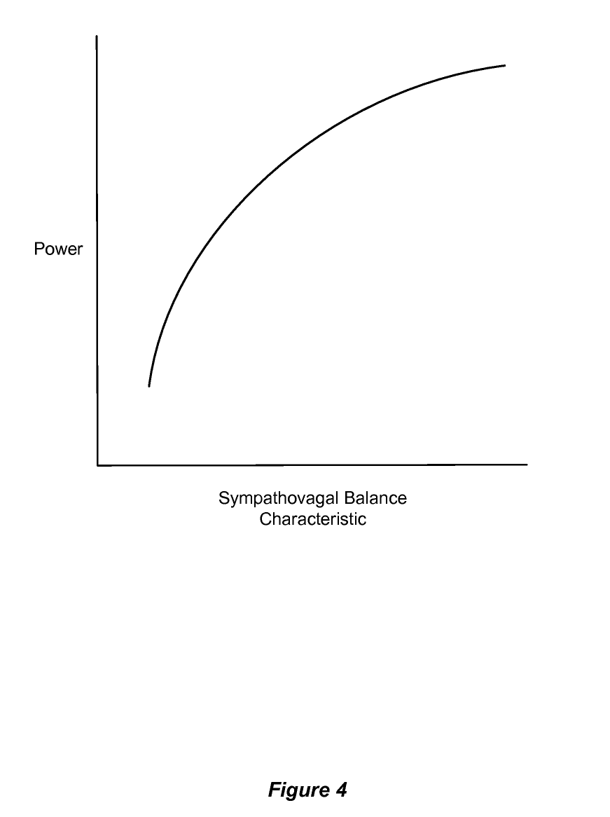

For example, FIG. 4 illustrates a chart which can be used to determine a parameter value for use in the method of FIG. 3 based on a measured characteristic of the ANS of the patient. Specifically, FIG. 4 illustrates a chart which can be used to determine a power setting for the electrical stimulus source generator. In this example, the power setting is determined based on a value related to Sympathovagal balance. In this example, a higher power setting is used for a higher calculated Sympathovagal balance value. Similar charts may be additionally or alternatively used to determine other parameters for programming the electrical stimulus source generator based on a measured characteristic of the ANS of the patient.

In step 330, an electrical stimulus is provided to the locations identified in step 310. For example, a needle may be inserted at each of the identified locations, where the needle is attached to the electrical stimulus source generator. In addition, a circuit completion path, such as a ground path, is provided by attaching a circuit completion electrode from the electrical stimulus source generator to the patient. The electrical stimulus is provided to the patient through the needles inserted at the locations identified in step 310 by the electrical stimulus source generator, which has been programmed with the parameter values of step 320.

Though the present invention is disclosed by way of specific embodiments as described above, those embodiments are not intended to limit the present invention. Based on the methods and the technical aspects disclosed above, variations and changes may be made to the presented embodiments by those skilled in the art without departing from the spirit and the scope of the present invention.

What is claimed is:

1. A method of caring for a patient, the method comprising:
measuring a first autonomic nervous system condition of the patient;
calculating a first autonomic dysfunction based on the measured first autonomic nervous system condition;
calculating a first sympathovagal balance based on the first measured autonomic nervous system condition;
treating the patient;
measuring a second autonomic nervous system condition of the patient;
calculating a second sympathovagal balance based on the second measured autonomic nervous system condition;
comparing the second sympathovagal balance with the first sympathovagal balance;
calculating a second autonomic dysfunction based on the measured second autonomic nervous system condition; and
comparing the second autonomic dysfunction with the first autonomic dysfunction.

2. The method of claim 1, further comprising:
measuring a third autonomic system condition of the patient;
calculating a third autonomic dysfunction based on the measured third autonomic system condition;
calculating a third sympathovagal balance based on the third measured autonomic system condition;
retreating the patient;
measuring a fourth autonomic system condition of the patient;
calculating a fourth sympathovagal balance based on the fourth measured autonomic system condition;
comparing the fourth sympathovagal balance with the third sympathovagal balance;
calculating a fourth autonomic dysfunction based on the measured fourth autonomic system condition; and
comparing the fourth autonomic dysfunction with the third autonomic dysfunction.

3. The method of claim 2, wherein measuring the first and second autonomic system condition of the patient and treating the patient are part of a first patient session, and wherein measuring the third and fourth autonomic system condition of the patient and retreating the patient are part of a second patient session.

4. The method of claim 2, further comprising:
calculating a fifth autonomic dysfunction based on one or more of the first and second measured autonomic system conditions;
calculating a sixth autonomic dysfunction based on one or more of the third and fourth measured autonomic system conditions; and
comparing the six autonomic dysfunction with the fifth autonomic dysfunction.

5. The method of claim 4, further comprising determining an efficacy of the treatment based on the comparison of the sixth autonomic dysfunction with the fifth autonomic dysfunction.

6. The method of claim 1, wherein calculating the first autonomic dysfunction comprises calculating a root of a sum of values, wherein one or more of the values is equal to a sum of difference values raised to an exponent, wherein the difference values are each equal to a difference of a first index value and a second index value, and wherein the first and second index values are each calculated based on the first autonomic nervous system condition.

7. The method of claim 6, wherein the difference values belong to a subset of a set of difference values, and wherein the subset comprises a plurality of difference values of the set which are sequential when the set of difference values is sorted by value.

8. The method of claim 7, wherein the boundaries between subsets are defined based on a second derivative of the set.

9. The method of claim 6, wherein the exponent is the inverse of the root.

10. The method of claim 1, wherein calculating sympathovagal balance comprises extracting a horizontal midpoint from a balance curve.

11. The method of claim 1, wherein treating the patient comprises electrically stimulating points on the patient to which the autonomic nervous system of the patient is sensitive.

12. The method of claim 11, wherein the power of the electrical stimulation is based on the first Sympathovagal balance.

13. The method of claim 1, wherein measuring each autonomic nervous system condition of the patient comprises measuring a heart rate of the patient over time.

14. The method of claim 1, wherein measuring each autonomic nervous system condition of the patient comprises recording first and second sets of data, wherein the first set of data is used to calculate autonomic dysfunction of the patient, and the second set of data is used to calculate a sympathovagal balance of the patient.

15. A method of caring for a patient, the method comprising:
measuring a first autonomic nervous system condition of the patient;
calculating a first sympathovagal balance based on the first measured autonomic nervous system condition;
treating the patient;
measuring a second autonomic nervous system condition of the patient;
calculating a second sympathovagal balance based on the second measured autonomic nervous system condition; and
comparing the second sympathovagal balance with the first sympathovagal balance.

16. The method of claim 15, further comprising:
measuring a third autonomic system condition of the patient;
calculating a third sympathovagal balance based on the third measured autonomic system condition;
retreating the patient;
measuring a fourth autonomic system condition of the patient;
calculating a fourth sympathovagal balance based on the fourth measured autonomic system condition; and
comparing the fourth sympathovagal balance with the third sympathovagal balance.

17. The method of claim 16, wherein measuring the first and second autonomic system condition of the patient and treating the patient are part of a first patient session, and wherein measuring the third and fourth autonomic system condition of the patient and retreating the patient are part of a second patient session.

18. The method of claim 16, further comprising:
calculating a fifth Sympathovagal balance based on one or more of the first and second measured autonomic system conditions;
calculating a sixth Sympathovagal balance based on one or more of the third and fourth measured autonomic system conditions; and
comparing the six Sympathovagal balance with the fifth Sympathovagal balance.

19. The method of claim 18, further comprising determining an efficacy of the treatment based on the comparison of the sixth Sympathovagal balance with the fifth Sympathovagal balance.

20. The method of claim 15, wherein calculating sympathovagal balance comprises extracting a horizontal midpoint from a balance curve.

21. The method of claim 15, wherein treating the patient comprises electrically stimulating points on the patient to which the autonomic nervous system of the patient is sensitive.

22. The method of claim 21, wherein the power of the electrical stimulation is based on the first Sympathovagal balance.

23. The method of claim 15, wherein measuring each autonomic nervous system condition of the patient comprises measuring a heart rate of the patient over time.

24. A method of caring for a patient, the method comprising:
measuring a first autonomic nervous system condition of the patient;
calculating a first autonomic dysfunction based on the measured first autonomic nervous system condition;
treating the patient;
measuring a second autonomic nervous system condition of the patient;
calculating a second autonomic dysfunction based on the measured second autonomic nervous system condition; and
comparing the second autonomic dysfunction with the first autonomic dysfunction.

25. The method of claim 24, further comprising:
measuring a third autonomic system condition of the patient;
calculating a third autonomic dysfunction based on the measured third autonomic system condition;
retreating the patient;
measuring a fourth autonomic system condition of the patient;
calculating a fourth autonomic dysfunction based on the measured fourth autonomic system condition;
comparing the fourth autonomic dysfunction with the third autonomic dysfunction.

26. The method of claim 25, wherein measuring the first and second autonomic system condition of the patient and treating the patient are part of a first patient session, and wherein measuring the third and fourth autonomic system condition of the patient and retreating the patient are part of a second patient session.

27. The method of claim 25, further comprising:
calculating a fifth autonomic dysfunction based on one or more of the first and second measured autonomic system conditions;
calculating a sixth autonomic dysfunction based on one or more of the third and fourth measured autonomic system conditions; and
comparing the six autonomic dysfunction with the fifth autonomic dysfunction.

28. The method of claim 27, further comprising determining an efficacy of the treatment based on the comparison of the sixth autonomic dysfunction with the fifth autonomic dysfunction.

29. The method of claim 24, wherein calculating the first autonomic dysfunction comprises calculating a root of a sum of values, wherein one or more of the values is equal to a sum of difference values raised to an exponent, wherein the difference values are each equal to a difference of a first index value and a second index value, and wherein the first and second index values are each calculated based on the first autonomic nervous system condition.

30. The method of claim 29, wherein the difference values belong to a subset of a set of difference values, and wherein the subset comprises a plurality of difference values of the set which are sequential when the set of difference values is sorted by value.

31. The method of claim 30, wherein the boundaries between subsets are defined based on a second derivative of the set.

32. The method of claim 29, wherein the exponent is the inverse of the root.

33. The method of claim 24, wherein treating the patient comprises electrically stimulating points on the patient to which the autonomic nervous system of the patient is sensitive.

34. The method of claim 24, wherein measuring each autonomic nervous system condition of the patient comprises measuring a heart rate of the patient over time.
